(19) United States
(12) Patent Application Publication
(10) Pub. No.: US 2025/0382567 A1
(43) Pub. Date: Dec. 18, 2025

(54) Acinetobacter SP. Strain Having Ability To Degrade Polyurethane and use Thereof

(71) Applicants: HYUNDAI MOTOR COMPANY, Seoul (KR); KIA CORPORATION, Seoul (KR); Industry Foundation of Chonnam National University, Gwangju (KR)

(72) Inventors: Choon Ho Lee, Hwaseong-si (KR); Dong Jun Lee, Hwaseong-si (KR); Young Ho Choi, Hwaseong-si (KR); Soo Jin Yeom, Gwangju (KR); Hyun Woo Kim, Gwangju (KR); Won Seok Chi, Gwangju (KR); Ki Jung Kim, Gwangju (KR)

(73) Assignees: HYUNDAI MOTOR COMPANY, Seoul (KR); KIA CORPORATION, Seoul (KR); Industry Foundation of Chonnam National University, Gwangju (KR)

(21) Appl. No.: 18/949,158

(22) Filed: Nov. 15, 2024

(30) Foreign Application Priority Data
Jun. 17, 2024 (KR) 10-2024-0077965

(51) Int. Cl. C12N 1/20; C08J 11/10; C12R 1/01

(52) U.S. Cl. CPC C12N 1/205; C08J 11/105; C12R 2001/01

(57) ABSTRACT

An Acinetobacter plasticovorax strain has high ability to degrade polyurethane. The strain is useful for reduction of environmental pollution caused by waste plastic, pretreatment for recycling, recycling, and bioconversion, and is further useful for carbon reduction for recycling of waste plastic.

### CROSS-REFERENCE TO RELATED APPLICATION

This application claims, under 35 U.S.C. § 119(a), the benefit of priority from Korean Patent Application No. 10-2024-0077965, filed on Jun. 17, 2024, the entire contents of which are incorporated herein by reference.

### REFERENCE TO AN ELECTRONIC SEQUENCE LISTING

This application contains a Sequence Listing that has been submitted electronically and is hereby incorporated by reference in its entirety. The Sequence Listing was created on Nov. 8, 2024, is named “202400087_Sequence list.xml”, and is 3,239 bytes in size.

### BACKGROUND

### (a) Technical Field

The present disclosure relates to an Acinetobacter plasticovorax strain having the ability to degrade polyurethane and the use thereof for biodegradation of waste polyurethane.

### (b) Background Art

Plastic wrappers and containers bring convenience to life due to excellent functionality and low prices, and contribute greatly to industrial development across industries. However, plastic waste, which is the most generated waste in modern society, is recognized as the largest source of environmental pollution, and technology capable of degrading the same is very lacking. Due to the depletion of petroleum, the main raw material for plastics, the need for recycling plastics discharged as waste is increasingly emerging.

Among plastics, polyurethane is a general term for polymer compounds with repeating urethane bonds, and has excellent heat resistance, wear resistance, and aging resistance and is thus used as a material for synthetic fibers, rubber, paints, adhesives, etc. Foam, manufactured by adding water during polymerization to induce foaming, is used as a material for bed mattresses. Moreover, polyurethane is composed of soft segments and hard segments, and by adjusting the ratio of these segments, various designs are possible, from elastic rubber to rigid engineering plastics. Soft segments refer to polyols having hydroxyl groups (—OH) at both ends, which are broadly classified into polyether and polyester, and hard segments refer to isocyanates having isocyanate groups (—N═C═O) at both ends.

Polyurethane is the 6^{th} most commonly used plastic in the world, and is currently replacing existing synthetic materials such as polyvinyl chloride (PVC), polystyrene, etc. However, due to the thermosetting properties of not melting when heated and the toxicity of some combustion products, polyurethane waste is difficult to recycle or destroy and is mostly landfilled or incinerated, causing environmental pollution by emitting toxic substances including carcinogens. Therefore, the importance of research into waste polyurethane treatment methods is increasing worldwide.

In addition to recent research to increase the safety and activity of the PETase enzyme that degrades PET, using protein engineering technology through a molecular approach to the relevant enzyme, an enzyme capable of decomposing 90% of what would take hundreds of years to degrade in the natural environment in just 16 hours has been developed.

However, polyurethane is not soluble in water or typical solvents, and is not affected by wide pH or temperature changes. Also, polyurethane is resistant to various types of biodeterioration due to the characteristics and relative proportions of components thereof. Attempts have been made to degrade waste polyurethane using chemicals, which is known to be a fairly inefficient method. Accordingly, polyurethane is difficult to pre-treat, and research results are insufficient compared to PET.

### SUMMARY OF THE DISCLOSURE

Therefore, the present disclosure has been made keeping in mind the problems encountered in the related art, and an object of the present disclosure is to provide an Acinetobacter plasticovorax strain having the ability to degrade polyurethane.

Another object of the present disclosure is to provide the use of an Acinetobacter plasticovorax strain for biodegradation of waste polyurethane.

Still another object of the present disclosure is to provide a composition for degrading polyurethane, including at least one selected from among an Acinetobacter plasticovorax strain, a culture of the strain, a concentrate of the culture, and a dried product of the culture.

Yet another object of the present disclosure is to provide a method of degrading plastic such as, for example, waste plastic, including treating waste plastic with at least one Acinetobacter plasticovorax strain, a culture of the strain, a concentrate of the culture, and/or a dried product of the culture.

The objects of the present disclosure are not limited to the foregoing. The objects of the present disclosure will be able to be clearly understood through the following description and to be realized by the means described in the claims and combinations thereof.

An embodiment of the present disclosure provides an Acinetobacter plasticovorax strain having polyurethane degradation activity.

The strain may be deposited under accession number KCTC15781BP.

The strain may include the 16S rRNA sequence represented by SEQ ID NO: 1.

The strain may remove at least one bond selected from among an N—H bond and a C—N bond from a urethane group (—NHCOO—) formed by polymerization reaction of a polyol compound and an isocyanate compound.

The strain may utilize polyurethane as a single carbon source.

Another embodiment of the present disclosure provides a composition for degrading polyurethane, including at least one selected from among an Acinetobacter plasticovorax strain, a culture of the strain, a concentrate of the culture, and a dried product of the culture.

Still another embodiment of the present disclosure provides a method of degrading plastic such as, for example, a waste plastic, including treating the waste plastic with at least one Acinetobacter plasticovorax strain, a culture of the strain, a concentrate of the culture, and/or a dried product of the culture.

The plastic, including a waste plastic may include polyurethane.

Treating the plastic (e.g., waste plastic) may be performed at 20° C. to 35° C.

### BRIEF DESCRIPTION OF THE DRAWINGS

The above and other features of the present disclosure will now be described in detail referring to certain exemplary embodiments thereof illustrated in the accompanying drawings, which are given hereinbelow by way of illustration only, and thus are not limitative of the present disclosure, and wherein:

FIG. 1 shows the optical density (OD₆₀₀) of colonies identified during screening of new polyurethane-degrading microorganisms according to an embodiment of the present disclosure;

FIG. 2 shows a phylogenetic diagram shown through comparison of the 16s rRNA sequence of an Acinetobacter plasticovorax strain;

FIG. 3 shows the growth of the Acinetobacter plasticovorax strain according to the present disclosure using polyurethane as a single carbon source as confirmed by the turbidity of liquid medium;

FIG. 4 shows changes in the chemical structure of amine (—CONH—) during degradation of polyurethane using Fourier-transform infrared spectroscopy (FT-IR) according to Experimental Example of the present disclosure;

FIG. 5 shows changes in the chemical structure of alkanoamine (—HN—COO—) during degradation of polyurethane using FT-IR according to Experimental Example of the present disclosure; and

FIG. 6 shows the chemical structure characteristics during degradation of polyurethane using X-ray photoelectron spectroscopy (XPS) according to Experimental Example of the present disclosure.

### DETAILED DESCRIPTION

The above and other objects, features and advantages of the present disclosure will be more clearly understood from the following preferred embodiments taken in conjunction with the accompanying drawings. However, the present disclosure is not limited to the embodiments disclosed herein, and may be modified into different forms. These embodiments are provided to thoroughly explain the disclosure and to sufficiently transfer the spirit of the present disclosure to those skilled in the art.

It will be understood that, although terms such as “first”, “second”, etc. may be used herein to describe various elements, these elements are not to be limited by these terms. These terms are only used to distinguish one element from another element. For instance, a “first” element discussed below could be termed a “second” element without departing from the scope of the present disclosure. Similarly, the “second” element could also be termed a “first” element. As used herein, the singular forms are intended to include the plural forms as well, unless the context clearly indicates otherwise.

It will be further understood that the terms “comprise”, “include”, “have”, etc., when used in this specification, specify the presence of stated features, integers, steps, operations, elements, components, or combinations thereof, but do not preclude the presence or addition of one or more other features, integers, steps, operations, elements, components, or combinations thereof. Also, it will be understood that when an element such as a layer, film, area, or sheet is referred to as being “on” another element, it may be directly on the other element, or intervening elements may be present therebetween. Similarly, when an element such as a layer, film, area, or sheet is referred to as being “under” another element, it may be directly under the other element, or intervening elements may be present therebetween.

Unless otherwise specified, all numbers, values, and/or representations that express the amounts of components, reaction conditions, polymer compositions, and mixtures used herein are to be taken as approximations including various uncertainties affecting measurement that inherently occur in obtaining these values, among others, and thus should be understood to be modified by the term “about” in all cases. Furthermore, when a numerical range is disclosed in this specification, the range is continuous, and includes all values from the minimum value of said range to the maximum value thereof, unless otherwise indicated. Moreover, when such a range pertains to integer values, all integers including the minimum value to the maximum value are included, unless otherwise indicated.

In the present specification, when a range is described for a variable, it will be understood that the variable includes all values including the end points described within the stated range. For example, the range of “5 to 10” will be understood to include any subranges, such as 6 to 10, 7 to 10, 6 to 9, 7 to 9, and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like. Also, for example, the range of “10% to 30%” will be understood to include subranges, such as 10% to 15%, 12% to 18%, 20% to 30%, etc., as well as all integers including values of 10%, 11%, 12%, 13% and the like up to 30%, and will also be understood to include any value between valid integers within the stated range, such as 10.5%, 15.5%, 25.5%, and the like.

An embodiment of the present disclosure relates to an Acinetobacter plasticovorax strain having polyurethane degradation activity.

In the present disclosure, the Acinetobacter plasticovorax strain has been deposited at the Korean Collection for Type Culture (KCTC) of the Korea Research Institute of Bioscience and Biotechnology on Jan. 25, 2024 under accession number KCTC15781BP.

In the present disclosure, the Acinetobacter plasticovorax strain may include the 16S rRNA sequence represented by SEQ ID NO: 1.

In the present disclosure, the Acinetobacter plasticovorax strain is able to remove at least one bond selected from among an N—H bond and a C—N bond from a urethane group (—NHCOO—) formed by polymerization reaction of a polyol compound and an isocyanate compound, and for example, is able to remove either or both of an N—H bond and a C—N bond.

In the present disclosure, the Acinetobacter plasticovorax strain is capable of growth using polyurethane as a single carbon source.

In the present disclosure, polyurethane may include at least one selected from among polyester polyurethane (PSPU) and polyether polyurethane (PEPU).

Another embodiment of the present disclosure relates to a composition for degrading polyurethane, including at least one selected from among an Acinetobacter plasticovorax strain, a culture of the strain, a concentrate of the culture, and a dried product of the culture.

Herein, a “culturing” or “culture” process may include any action performed for growth of microorganisms under appropriately artificially controlled environmental conditions. A “culture,” which is the result of culturing, means including a “fermented product,” and an example thereof may include a culture fluid itself obtained by culturing in medium (i.e., a culture fluid itself containing bacteria) or a processed product derived from the culture fluid, such as a processed product obtained by subjecting the culture fluid to heat treatment, etc.

The culture temperature of the microorganism according to the present disclosure may be about 20° C. to 35° C. or about 25° C. to 30° C., but is not limited thereto and may be adjusted to various ranges in consideration of the growth rate of the microorganism.

The culture time of the microorganism according to the present disclosure may be about 1 day or more, about 2 days or more, about 3 days or more, about 4 days or more, or about 5 days or more, but is not limited thereto and may be adjusted to various ranges in consideration of the growth rate of the microorganism.

The composition according to the present disclosure may include, in addition to an active ingredient, such as a strain, a culture thereof, a concentrate of the culture, a dried product of the culture, and/or a culture supernatant of the strain, a culture containing bacterial cells, an extract of the bacterial cells, and a concentrate liquid, a concentrate, and a dried product thereof, and also a dilution liquid, a dilution, etc. as necessary, and may include a culture in any state obtained by treating the culture fluid or the culture. In embodiments, the composition in accordance with the disclosure comprises an amount of a biologically pure microbial strain (e.g., bacterial strain such as, for example, Acinetobacter plasticovorax) as disclosed herein. In some embodiments, the composition comprises an amount of a biologically pure Acinetobacter plasticovorax strain deposited under accession number KCTC15781BP. In some embodiments the composition comprises an amount of a biologically pure Acinetobacter plasticovorax strain comprising a 16S rRNA sequence of SEQ ID NO:1. In accordance with any of the above embodiments, the amount of the biologically pure strain can be in an amount effective to degrade and/or metabolize a plastic such as, for example, polyurethane.

In the present disclosure, the culture method, extraction method, separation method, concentration method, drying method, dilution method, etc. of the strain are not particularly limited.

The medium for culturing the strain may include sugars, yeast extract, etc., which are typically contained in culture medium. As a culture method, a variety of general aerobic or anaerobic methods may be used as appropriate, and after culturing the strain, the culture or the supernatant thereof may be concentrated, dried, or diluted as necessary.

Also, the supernatant of the culture and the bacterial cells may be separated from each other using centrifugation or membrane separation, and thus the bacterial cells may be recovered in a concentrated state. Moreover, the bacterial cells may be subjected to ultrasonic treatment or enzymatic treatment to extract components from the bacterial cells, or the culture, the supernatant thereof, the bacterial cells or the extract thereof, etc. may be dried. These may be used as active ingredients in the composition including the strain of the present disclosure.

The composition including the strain according to the present disclosure may be used to degrade polyurethane.

As used herein, the term “degradation” may refer to biodegradation using microorganisms or microbial enzymes, and specifically may be a concept including biodeterioration, which is a general term not only for superficial degradation and deterioration of plastic due to the chemical and physical actions of microorganisms but also for changes in the chemical, physical, and mechanical properties of plastic, and biofragmentation, which indicates a depolymerization step in which plastic subjected to biodeterioration is converted from a polymer into a monomer by enzymes or free radicals formed by microorganisms. For example, products resulting from biodegradation of polyurethane may include aromatic amines formed by cleavage of urethane bonds that release hard segments.

Still another embodiment of the present disclosure relates to a method of degrading waste plastic, including treating waste plastic with at least one Acinetobacter plasticovorax strain, a culture of the strain, a concentrate of the culture, and/or a dried product of the culture.

In the present disclosure, the plastic such as, for example, waste plastic may include polyurethane, but is not particularly limited thereto, and may further include polyethylene terephthalate (PET), polyvinyl chloride (PVC), polystyrene (PS), polypropylene (PP), polyethylene (PE), etc.

In the present disclosure, solid plastic or waste plastic may be frozen with liquid nitrogen and then pulverized.

In the present disclosure, the plastic or waste plastic may be pretreated with a chemical or an additive such as an oxidation accelerator, a combustion reducer, or a pre-modifier, but the present disclosure is not limited thereto. Pretreatment may serve to partially change the biodegradation rate by changing the functional group or hydrophilicity/hydrophobicity of the plastic, and the main aim thereof may be reducing degradation resistance of the plastic through mechanisms such as supplying an additional carbon source or increasing surface hydrophilicity of plastic (e.g., waste plastic).

In the present disclosure, treating the plastic (e.g., waste plastic) may be performed at room temperature in the range of about 20° C. to 35° C., about 20° C. to 30° C., about 25° C. to 35° C., or about 25° C. to 30° C., but the present disclosure is not limited thereto.

A better understanding of the present disclosure may be obtained through the following example and experimental example. The example and experimental example are merely set forth to illustrate the present disclosure, and are not to be construed as limiting the scope of the present disclosure.

### 1. Example: Selection and Identification of Strain

### 1.1. Purchasing of Medium and Physical Pretreatment of Polyurethane

M9 minimal broth medium, Nutrient Broth (NB) medium, Luria Bertani (LB) broth, and Yeast Extract were purchased from MB Cell (2nd floor, Kisan Building, 11 Yangjaecheon-ro 31-gil, Seocho-gu, Seoul), and a trace element solution (composition: 14.7 mg of MnCl₂·4H₂O, 0.78 g of CaCl₂, 1.62 mg of FeCl₃·6H₂O, 24.6 mg of CuSO₄·5H₂O, 21.6 mg of NiCl₂·6H₂O, and 21.8 mg/l of CoCl₂·6H₂O) was prepared.

Polyurethane was supplied by Hyundai Motor Company. Polyurethane was frozen with liquid nitrogen, pulverized in a mortar and pestle, and sterilized in 100% ethanol for one day.

### 1.2. Screening of New Polyurethane-Degrading Microorganism

A sample obtained from a sewage treatment plant in Bonghwa-gun, Gyeongsangbuk-do was filtered with WHATMAN filter paper (pore size: 11 m), and 1 ml thereof was spread on M9 minimal solid medium containing 10 mg/ml polyurethane and 0.002 g/l yeast extract, followed by culture in an incubator at 28° C. for 14 days. In order to confirm whether the resulting colonies were able to grow using polyurethane as a single carbon source, streaking was performed on solid medium containing the same composition as above, followed by culture in an incubator at 28° C. for 14 days.

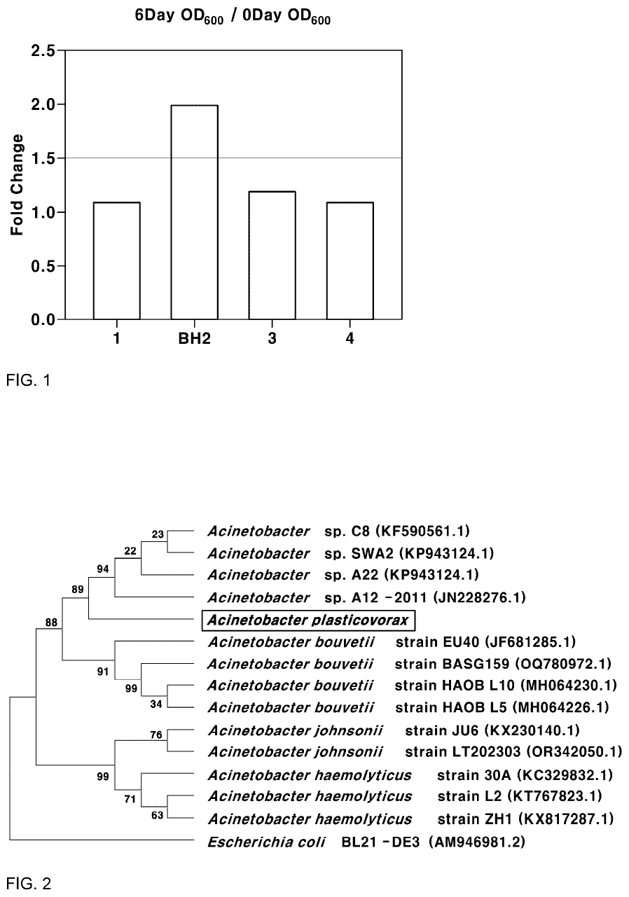

The resulting colonies were inoculated into LB liquid medium and cultured in an incubator at 28° C. for one day with shaking at 200 rpm. Then, to remove the carbon source, centrifugation at 4,000 rpm for 20 minutes and washing with M9 minimal liquid medium were performed. The carbon source removal process was repeated twice. The microorganisms washed with M9 minimal medium were inoculated at 10% in 3 ml of M9 minimal liquid medium containing 10 mg/ml polyurethane and cultured at 28° C. for 6 days at 200 rpm. The results thereof are shown in FIG. 1. Microbial growth was confirmed using optical density (OD₆₀₀), and one strain showing the highest growth was selected.

### 1.3. Identification and Phylogenetic Tree of Bacteria

The selected strain was obtained by streaking on NB solid medium. For identification of the strain, solid medium was sent to Solgent, Daejeon, Korea, and 16S rRNA sequencing was requested and analyzed using universal primers 27F (5′-AGAGTTTGATCCTGGCTCAG-3′) and 1492R (5′-GGTTACCTTGTTACGACTT-3′). A phylogenetic tree was constructed in MEGA X (10.2.6 version) software by performing 1,000 Bootstrap replications by the Maximum-likelihood (ML) method using the GTR+I+G model, utilizing gene sequence data from the NCBI gene database.

Thereby, based on the 16S rRNA gene sequence, the polyurethane-degrading strain BH2 isolated from sewage sludge was confirmed to be 99.64% identical to the Acinetobacter sp. A12-2011 strain (NCBI Reference Sequence: JN228276.1). Moreover, when constructing and analyzing the phylogenetic tree, this new polyurethane-degrading strain showed a close relationship in the phylogenetic tree of Acinetobacter sp., and was named Acinetobacter plasticovorax to identify the same as a new species and deposited at the Korea Research Institute of Bioscience and Biotechnology on Jan. 25, 2024 (Accession number: KCTC15781BP). The 16S rRNA sequence of the Acinetobacter plasticovorax strain is shown in Table 1 below. Furthermore, a phylogenetic diagram obtained through comparison of the 16s rRNA sequence of the Acinetobacter plasticovorax strain is shown in FIG. 2.

TABLE 1

| SEQ ID | | | |
|---|---|---|---|
| NO: | Name | Sequence | Note |
| 1 | 16S rRNA_ | AGTCGAGCGGGGAGATGTAGC | 1401 |
| | Acinetobacter | TTGCTACATTTCCTAGCGGCG | bp |
| | plasticovorax | GACGGGTGAGTAATGCTTAGG | |
| | | AATCTGCCTATTAGTGGGGGA | |
| | | CAACGTTTCGAAAGGAACGCT | |
| | | AATACCGCATACGCCCTACGG | |
| | | GGGAAAGCAGGGGCTCTTCGG | |
| | | ACCTTGCGCTAATAGATGAGC | |
| | | CTAAGTCAGATTAGCTAGTTG | |
| | | GTGGGGTAAAGGCCTACCAAG | |
| | | GCGACGATCTGTAGCGGGTCT | |
| | | GAGAGGATGATCCGCCACACT | |
| | | GGGACTGAGACACGGCCCAGA | |
| | | CTCCTACGGGAGGCAGCAGTG | |
| | | GGGAATATTGGACAATGGGGG | |
| | | GAACCCTGATCCAGCCATGCC | |
| | | GCGTGTGTGAAGAAGGCCTTT | |
| | | TGGTTGTAAAGCACTTTAAGC | |
| | | GAGGAGGAGGCTCCTATAGAT | |
| | | AATACCTATAGTGAGTGGACG | |
| | | TTACTCGCAGAATAAGCACCG | |
| | | GCTAACTCTGTGCCAGCAGCC | |
| | | GCGGTAATACAGAGGGTGCGA | |
| | | GCGTTAATCGGATTTACTGGG | |
| | | CGTAAAGCGTACGTAGGCGGC | |
| | | TTTTTAAGTCGGATGTGAAAT | |
| | | CCCTGAGCTTAACTTAGGAAT | |
| | | TGCATTCGATACTGGGAAGCT | |
| | | AGAGTATGGGAGAGGATGGTA | |
| | | GAATTCCAGGTGTAGCGGTGA | |
| | | AATGCGTAGAGATCTGGAGGA | |
| | | ATACCGATGGCGAAGGCAGCC | |
| | | ATCTGGCCTAATACTGACGCT | |
| | | GAGGTACGAAAGCATGGGGAG | |
| | | CAAACAGGATTAGATACCCTG | |
| | | GTAGTCCATGCCGTAAACGAT | |
| | | GTCTACTAGCCGTTGGGGCCT | |
| | | TTGAGGCTTTAGTGGCGCAGC | |
| | | TAACGCGATAAGTAGACCGCC | |
| | | TGGGGAGTACGGTCGCAAGAC | |
| | | TAAAACTCAAATGAATTGACG | |
| | | GGGGCCCGCACAAGCGGTGGA | |
| | | GCATGTGGTTTAATTCGATGC | |
| | | AACGCGAAGAACCTTACCTGG | |
| | | TCTTGACATAGTAAGAACTTT | |
| | | CCAGAGATGGATTGGTGCCTT | |
| | | CGGGAACTTACATACAGGTGC | |
| | | TGCATGGCTGTCGTCAGCTCG | |
| | | TGTCGTGAGATGTTGGGTTAA | |
| | | GTCCCGCAACGAGCGCAACCC | |
| | | TTTTCCTTATTTGCCAGCGGG | |
| | | TTAAGCCGGGAACTTTAAGGA | |
| | | TACTGCCAGTGACAAACTGGA | |
| | | GGAAGGCGGGGACGACGTCAA | |
| | | GTCATCATGGCCCTTACGACC | |
| | | AGGGCTACACACGTGCTACAA | |
| | | TGGTCGGTACAAAGGGTTGCT | |
| | | ACCTAGCGATAGGATGCTAAT | |
| | | CTCAAAAAGCCGATCGTAGTC | |
| | | CGGATTGGAGTCTGCAACTCG | |
| | | ACTCCATGAAGTCGGAATCGC | |
| | | TAGTAATCGCGGATCAGAATG | |
| | | CCGCGGTGAATACGTTCCCGG | |
| | | GCCTTGTACACACCGCCCGTC | |
| | | ACACCATGGGAATTTGTTGCA | |
| | | CCAGAAGTAGGTAGTCTAACC | |
| | | GCAAGGAGGACGCTA | |

### 1.4. Confirmation of Growth in Liquid Medium Containing Polyurethane as Single Carbon Source

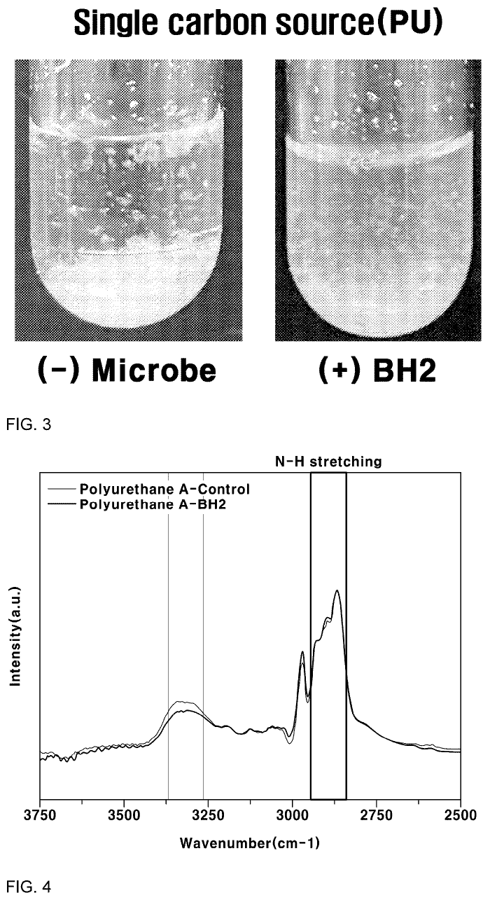

The Acinetobacter plasticovorax strain was inoculated into LB liquid medium and cultured in an incubator at 28° C. for one day with shaking at 200 rpm. Then, to remove the carbon source, centrifugation at 4,000 rpm for 20 minutes and washing with M9 minimal liquid medium were performed. The carbon source removal process was repeated twice. Next, in the control group, liquid medium contained polyurethane alone, and in the experimental group, the Acinetobacter plasticovorax strain was added. 280 ml of the washed strain, corresponding to an optical density of 0.2, was placed in a 300 ml biochemical oxygen demand (BOD) bottle containing 10 mg/ml polyurethane. Culture was carried out in an incubator at 28° C. for 6 days with shaking at 140 rpm, and the concentration of dissolved oxygen (DO) was measured every 3 days. The change in turbidity after measurement is shown in FIG. 3, and the results of measurement of dissolved oxygen are shown in Table 2 below.

TABLE 2

| | Initial DO | DO after 3 days | DO after 6 days |
|---|---|---|---|
| Classification | (mg/l) | (mg/l) | (mg/l) |
| Acinetobacter | 7.305 ± 0.005 | 0.007 ± 0.001 | 0.007 ± 0.001 |
| plasticovorax | | | |

As can be seen in FIG. 3 and Table 2, the amount of dissolved oxygen was decreased to 0.007 mg/l on day 3 from 7.305 mg/l on day 0. Based on these results, the Acinetobacter plasticovorax strain of the present disclosure was confirmed to use polyurethane as a single carbon source.

### 2. Experimental Example: Analysis of Chemical Structure Characteristics During Degradation of Polyurethane

The Acinetobacter plasticovorax strain was inoculated in an amount corresponding to an optical density of 0.2 into medium containing 10 mg/ml polyurethane as a single carbon source and cultured at 28° C. for 30 days at 200 rpm. Thereafter, the culture fluid and polyurethane were separated from each other using WHATMAN filter paper, followed by washing three times with 100% ethanol. The washed polyurethane was completely dried in a vacuum oven at 60° C., and Fourier-transform infrared spectroscopy (FT-IR) and X-ray photoelectron analysis (XPS) were performed.

Fourier-transform infrared spectroscopy (FT-IR, IRAffinity-IS, Shimadzu, Kyoto, Japan) was used to measure the functional groups of polyurethane in the wavelength range of 4,000-500 cm⁻¹ with a resolution of 4 cm⁻¹.

### 2.1. FT-IR Results

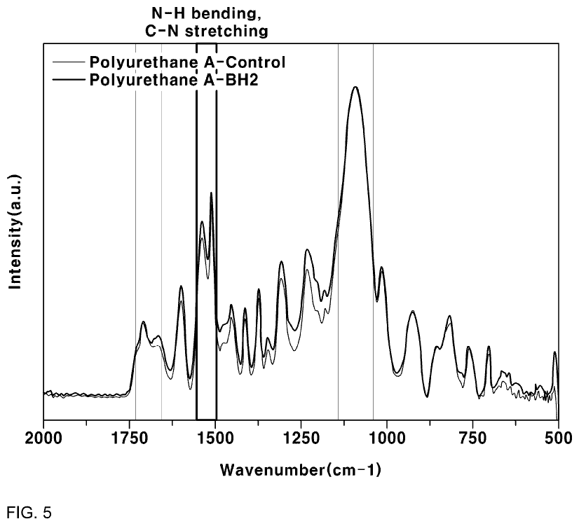

The FT-IR measurement results are shown in FIGS. 4 and 5.

As can be seen in FIGS. 4 and 5, in the polyurethane biologically degraded by culturing the Acinetobacter plasticovorax strain for 30 days, the peaks (N—H and C—N stretching) corresponding to the N—H and C—N bonds decreased compared to the control group. This is deemed to be because the N—H and C—N bonds of the polymer chain decreased through biodegradation of polyurethane by Acinetobacter plasticovorax.

Specifically, in the polyurethane of the experimental group (treated with the strain of the present disclosure), N—H and C—N stretching decreased compared to the control group, which is deemed to be because the N—H sites {amine (—CONH—) and alkanoamine (—HN—COO—)} in the chemical structural formula (corresponding to the hard segment) of the polyurethane polymer represented below disappeared, resulting in a decrease in total N—H and C—N bonds (indicated by ovals in the chemical structural formula below).

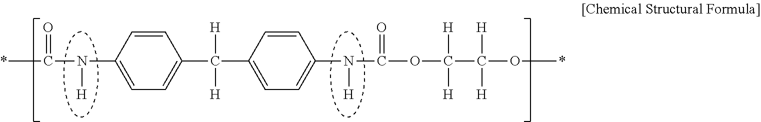

### 2.2. XPS Results

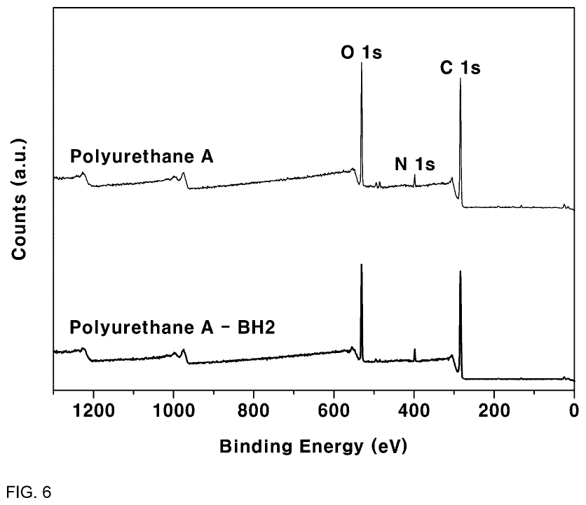

The results of X-ray photoelectron spectroscopy (XPS) are shown in FIG. 6 and Table 3 below.

TABLE 3

| | Atomic % | Atomic % |
|---|---|---|
| Classification | (control) | (Acinetobacter plasticovorax) |
| C 1 s | 72.26 | 74.14 |
| N 1 s | 3.73 | 3.79 |
| O 1 s | 24.01 | 22.07 |

As can be seen in FIG. 6 and Table 3, in the polyurethane biologically degraded by culturing the Acinetobacter plasticovorax strain for 30 days, the amounts of nitrogen and oxygen relative to the amount of carbon contained in the polymer chain decreased compared to the control group. This means that, consistent with the FT-IR results above, there was a change in the polyurethane polymer chain and the oxygen and nitrogen contained in the polymer chain decreased.

Consequently, the Acinetobacter plasticovorax strain of the present disclosure is capable of growth using polyurethane as a single carbon source and has high ability to degrade polyurethane, confirming the usefulness thereof for biodegradation of polyurethane in plastics, including waste plastic.

### Biological Deposit Information

Name of depository institution: Korean Collection for Type Culture (KCTC)

Accession number: KCTC15781BP

Accession date: 20240125

As is apparent from the above description, an Acinetobacter plasticovorax strain according to the present disclosure has high ability to degrade polyurethane, and is thus useful for reduction of environmental pollution caused by plastics, (e.g., microplastics, waste plastic, etc.), pretreatment for recycling, recycling, and bioconversion, and is further useful for carbon reduction for recycling of waste plastic.

In particular, the use of the microorganism according to the present disclosure enables effective degradation of polyurethane without generating harmful substances through a simple process, unlike a conventional polyurethane degradation process that requires complex pretreatment and degradation processes.

The effects of the present disclosure are not limited to the foregoing. It should be understood that the effects of the present disclosure include all effects that can be inferred from the description of the present disclosure.

What is claimed is:

1. An Acinetobacter plasticovorax strain having polyurethane degradation activity.

2. The strain of claim 1, wherein the strain is deposited under accession number KCTC15781BP.

3. The strain of claim 1, wherein the strain comprises a 16S rRNA sequence represented by SEQ ID NO: 1.

4. The strain of claim 1, wherein the strain removes at least one bond selected from an N—H bond and a C—N bond from a urethane group (—NHCOO—) formed by polymerization reaction of a polyol compound and an isocyanate compound.

5. The strain of claim 1, wherein the strain utilizes polyurethane as a single carbon source.

6. A composition for degrading polyurethane, comprising at least one of an Acinetobacter plasticovorax strain, a culture of the strain, a concentrate of the culture, and a dried product of the culture.

7. A method of degrading a waste plastic, comprising treating the waste plastic with at least one of an Acinetobacter plasticovorax strain, a culture of the strain, a concentrate of the culture, and a dried product of the culture.

8. The method of claim 7, wherein the waste plastic comprises polyurethane.

9. The method of claim 7, wherein the strain is deposited under accession number KCTC15781BP.

10. The method of claim 7, wherein the strain comprises a 16S rRNA sequence represented by SEQ ID NO: 1.

11. The method of claim 7, wherein the strain removes at least one bond selected from among an N—H bond and a C—N bond from a urethane group (—NHCOO—) formed by polymerization reaction of a polyol compound and an isocyanate compound.

12. The method of claim 7, wherein the strain utilizes polyurethane as a single carbon source.

13. The method of claim 7, wherein treating the waste plastic is performed at 20° C. to 35° C.

14. A composition comprising an amount of a biologically pure culture of the Acinetobacter plasticovorax strain of claim 2, wherein the amount is effective for degrading polyurethane.

15. A composition comprising an amount of a biologically pure culture of the Acinetobacter plasticovorax strain of claim 3, wherein the amount is effective for degrading polyurethane.

16. The composition of claim 14, wherein the biologically pure culture of the strain comprises a concentrate of the biologically pure culture or a dried product of the biologically pure culture.

17. The composition of claim 15, wherein the biologically pure culture of the strain comprises a concentrate of the biologically pure culture or a dried product of the biologically pure culture.


SEQUENCE LISTING (1 sequence)

SEQ ID NO: 1 - RNA, 1401 nt, Acinetobacter plasticovorax
  source 1..1401: mol_type = rRNA
agtcgagcggggagatgtagcttgctacatttcctagcggcggacgggtgagtaatgcttaggaatctgcctattagtgggggacaacgtttcgaaaggaacgctaataccgcatacgccctacgggggaaagcaggggctcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttttggttgtaaagcactttaagcgaggaggaggctcctatagataatacctatagtgagtggacgttactcgcagaataagcaccggctaactctgtgccagcagccgcggtaatacagagggtgcgagcgttaatcggatttactgggcgtaaagcgtacgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggaatttgttgcaccagaagtaggtagtctaaccgcaaggaggacgcta